(19) United States
(12) Patent Application Publication
(10) Pub. No.: US 2002/0164610 A1
(43) Pub. Date: Nov. 7, 2002

(54) Method for typing a cell

(76) Inventors: Carol G. Leggett, Cairo, GA (US); Ellyn Whitehouse, Wacissa, FL (US); Robert H. Reeves, Tallahassee, FL (US)

Correspondence Address: Stanley A. Kim, Akerman, Senterfitt & Eidson, P.A., 222 Lakeview Avenue, Suite 400, P.O. Box 3188, West Palm Beach, FL, 33402-3188, US

(21) Appl. No.: 10/001,048

(22) Filed: Nov. 1, 2001

Related U.S. Application Data
(62) Division of application No. 08/461,210, filed on Jun. 5, 1995, now Pat. No. 6,395,475.

(51) Int. Cl. C12Q 1/68

(52) U.S. Cl. 435/6

(57) ABSTRACT

A method for typing a cell includes the steps of: (a) providing a cell of unknown type, the cell of unknown type having a nucleic acid forming a portion of the cell's genome being IGR1, IGR2, ETS1, or ETS2; (b) isolating the nucleic acid from the cell of unknown type; (c) amplifying the nucleic acid by PCR to form a PCR product; (d) digesting the PCR product with a restriction endonuclease to form a plurality of restriction fragments; (e) separating the plurality of restriction fragments to generate a restriction pattern; and (f) comparing the restriction pattern generated in step (e) with a restriction pattern of a cell of known type.

### CROSS-REFERENCE TO RELATED APPLICATIONS

The present application is a divisional of and claims the benefit of U.S. patent application Ser. No. 08/461,210, entitled “SEMIAUTOMATED METHOD FOR FINGERPRINTING BACTERIAL DNA,” filed Jun. 5, 1995. All patent applications, patents, and other references are incorporated herein by reference to the extent they do not conflict with this patent application.

### BACKGROUND

Nosocomial (hospital-based) infections have become one of the most serious problems in infectious disease. Staphylococcus aureus is exceeded only by Escherichia coli as a leading cause of nosocomial infections. See, for example, Brumfitt, W. et al., Drugs Exptl. Clin. Res., 16:205-214 (1990). One type of S. aureus, methicillin-resistant S. aureus (MRSA), is of a particular interest because it is resistant to all penicillin-based antibiotics.

Patients in the intensive care unit are very susceptible to bacterial infections, due to interventions such as respiratory tubes and indwelling catheters. E. coli and S. aureus, if introduced into surgical wounds, the blood stream or the urinary tract, cause serious, sometimes life-threatening infections. The Infection Control Committees of most hospitals are constantly fighting this problem. There is a no easy solution to the problem, but a partial solution in most “nosocomial outbreaks” is simply identifying the source of the infection. That is, is the infectious agent coming from a common source (e.g. an infected nurse or doctor, or an instrument such as a respirator) or is there some other reason for the sudden emergence of a single type of bacterial infection.

Hospital laboratories can quickly identify the infectious agent (e.g., S. aureus), but they do not have the ability to determine whether a single strain of the organism is causing the outbreak (and therefore a possible common source) or if several different strains are responsible for the outbreak. At the present time most of these outbreaks can only be characterized in retrospect, since the outbreak is over before the bacterial isolates can be identified. For example, in an outbreak of S. aureus in a hospital nursery, the isolates can be identified in a matter of 1 or 2 days, but the strain identification usually takes weeks or even months, because the strains are still analyzed by slow culture-based methods which are labor intensive.

Current methods of strain typing bacteria include phage typing, plasmid analysis, and antibiotic susceptibility (biotyping). See, for example, Zuccarelli, A. et al.: J. Clin. Microbiol., 28:97-102 (1990); Tokue, Y. et al.: Tohokin J. Exp. Med., 163:31-37 (1991); Coia, J. et al.: J. Med. Microbiol., 31:125-132 (1990); Pennington, T. et al.: J. Clin. Microbiol., 29:390-392 (1991); Tveten, Y. et al.: J. Clin. Microbiol., 29:110-1105 (1991); Fluit, A. et al.: Eur. J. Clin. Microbiol. Infect. Dis., 9:605-608 (1990); Thomson-Carter, F. et al.: J. Gen. Microbiol., 135:2093-2097 (1989); and Preheim, L. et al.: Eur. J. Clin. Microbiol. Infect. Dis., 10:428-436 (1991). These methods, currently used by the Centers for Disease Control, are laborious, time consuming (approximately one month) and often yield inconclusive results.

Consequently, there is a serious need in the medical community for means to not only identify the infectious agents, but also to rapidly characterize the strain or strains involved so that effective measures may be timely employed.

### SUMMARY OF THE PRESENT INVENTION

In brief, the present invention alleviates and overcomes certain of the above-mentioned drawbacks of the present state of the art through the discovery of novel methods and kits for rapidly fingerprinting DNA to identify prokaryotic and eukaryotic species, subspecies, and especially stains or individuals of the subspecies. As to prokaryotic organisms, the present invention is especially suited for identifying different bacterial strains involved in, for example, nosocomial infections, since the methods and kits are believed to be sensitive enough to detect differences between, for example, bacterial isolates of the same species. With respect to eukaryotes, the present invention contemplates identifying, for instance, species, subspecies, and the differences between the individuals of the subspecies, such as pedigrees.

Generally speaking, the present invention involves the use of polymerase chain reaction (PCR) technology and restriction fragment length polymorphism analysis of genomic DNA preferably containing numerous gene clusters, such as ribosomal RNA (rRNA) gene clusters. In accordance with the present invention, a specific DNA fragment in a gene cluster region, such as the rRNA intergene region, is amplified by PCR using two universal oligonucleotide primers. This area of the genome is generally ideal for such a procedure because a highly variable spacer region is flanked by two highly conserved genes which may be used as primer sites. Before PCR amplification, however, the oligonucleotides are preferably fluorescently labeled. While it is preferable to label the 5′ end primers, the 3′ end primers or both the 5′ and 3′ end primers, as well as the individual nucleotides utilized during PCR may be labeled. Because the 5′ end of each cluster is preferably fluorescently-labeled, each fragment is represented as an individual peak on a waveform pattern. Therefore, since E. coli has seven clusters within its genome, multiple peaks will be present on the waveform pattern. The labeled PCR product may then be cleaved with a variety of restriction endonucleases and electrophoresed on an automated DNA sequencer.

Thus, the methods and kits of the present invention generally depend upon rapid, semiautomated DNA analysis, and more particularly, upon a type of DNA fingerprinting of multiple segments of DNA, such as the ribosomal RNA gene clusters, that are common to particular prokaryotic or eukaryotic species. Moreover, the methods and kits are believed to be most beneficial in a clinical laboratory because they allow for rapid strain identification of pathogenic bacteria. The DNA fingerprinting methods and kits of the present invention are also believed to be more definitive than currently practiced methodologies, since genomic DNA is used.

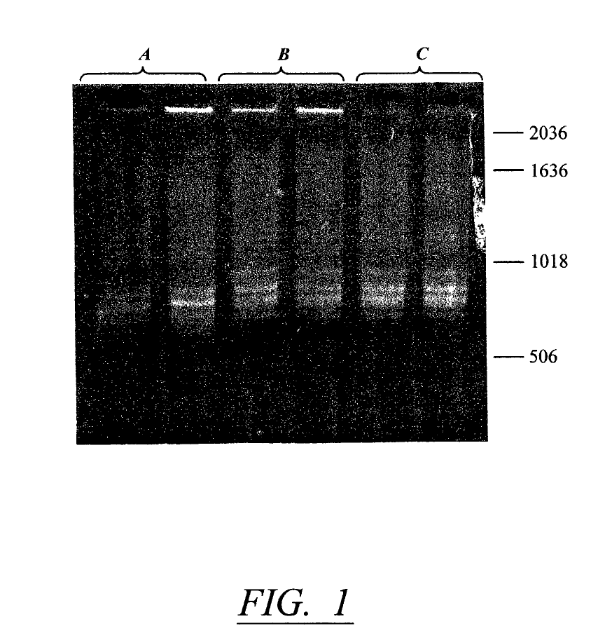

One main advantage of the methods and kits of the present invention is the speed with which results are obtained. A preliminary screen by agarose gel electrophoresis of a PCR product can be completed within five to six hours after receiving a sample, such as a hospital isolate. More particularly, the differences in the intergene region are detected on, for example, a 2% agarose gel, by banding patterns following PCR. Identical strains will exhibit similar banding patterns, whereas strains from other sources or of different types will differ in band intensity and fragment size (as demonstrated in FIG. 1).

The preliminary screen can then be confirmed in approximately 24 hours by restriction fragment length polymorphism (RFLP) analysis on an automated sequencer. For example, more definitive analyses are performed on the Applied Biosystems, Inc. (Foster City, Calif.) vertical electrophoresis unit with the Genescan software. Following PCR, restriction endonuclease digestions are performed and the cleaved fragments are loaded directly onto the electrophoresis unit. Although a multitude of restriction endonucleases are available for performing this assay, HhaI and HindIII are believed to be the most informative concerning at least prokaryote typing.

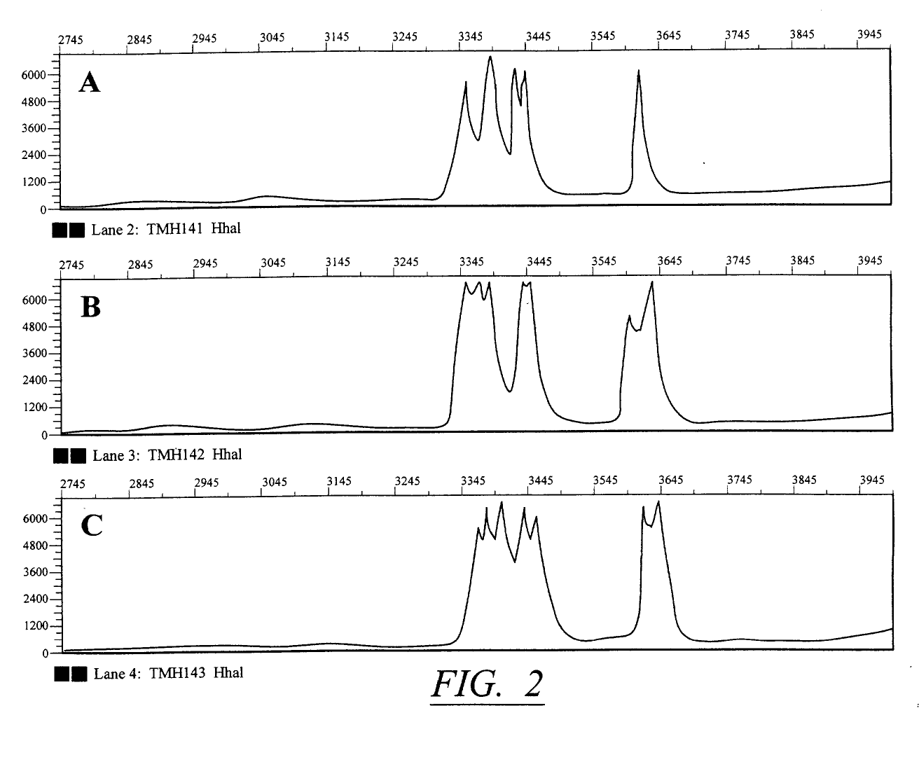

MRSA digested with HhaI consistently exhibit patterns with fragment lengths of 276, 278, 283, and 299 base pairs (see FIG. 2). Although fragment sizes are consistent among strains, a remarkable difference in band intensity is noted for each individual stain. Band intensity is measured by the software as peak area and may be converted to gene dosage (gene copy number) by using a reference gene during the electrophoresis run. It is our belief that differences between strains or individuals of subspecies may be determined based on gene dosage of each fragment at these given positions. A gene which may be universal among eubacteria and may be useful as a standard gene for calculating gene dosages has also been identified. This gene is dna A.

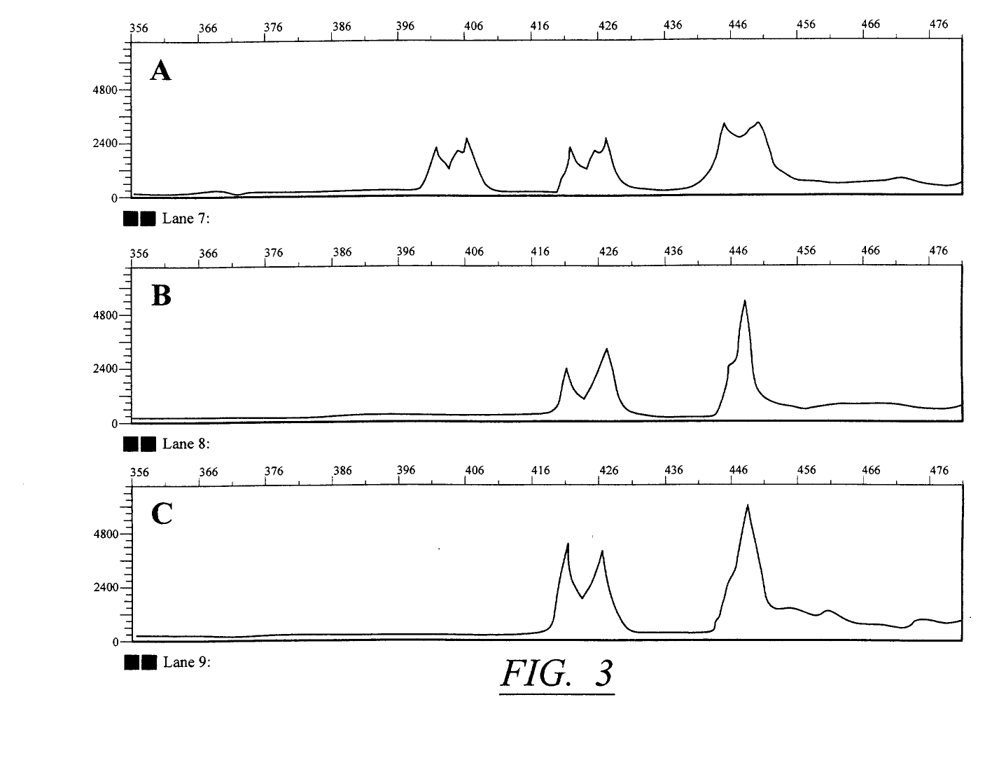

HindIII restriction endonuclease digestions of MRSA have been used to corroborate HhaI data. Typical HindIII patterns exhibit two to three fragment groupings with varying fragment lengths (see FIG. 3). Each grouping is typically positioned fourteen to twenty bases away from its neighboring group. Strain determinations are based on the number of groupings present and fragment length. The speed of these procedures provides, for example, infection control personnel adequate information to contain and prevent the spread of nosocomial infections, rather than having analyses done retrospectively.

Accordingly, it can now be appreciated that the present invention is believed to provide a solution to identifying differences between species, subspecies, and strains of prokaryotic organisms and individuals of subspecies of higher life forms.

The above features and advantages will be better understood with reference to the FIGS., Examples and Detailed Description set out herein below. It will also be understood that the methods and kits of the present invention are exemplary only and are not to be regarded as limitations of this invention.

### BRIEF DESCRIPTION OF THE FIGS.

Reference is now made to the accompanying FIGS. in which are shown characteristics corresponding to the unique DNA fingerprinting data generated via the methods of the present invention from which certain of their novel features and advantages will be apparent:

FIG. 1 is a preliminary screen of amplified intergene regions of clinical MRSA isolates. PCR products were electrophoresed on a 2% agarose gel and stained with ethidium bromide. Lanes 1 and 2, isolate A; lanes 3 and 4, isolate B; lanes 5 and 6, isolate C.

FIG. 2 is a comparison of electrophoretograms of the amplified intergene region of three clinical isolates cleaved with HhaI. Similarities between isolates B and C indicate they are from a common source, whereas isolate A originates from an independent source.

FIG. 3 is a comparison of electrophoretograms of the amplified intergene region of three clinical isolates cleaved within HindIII. Similarities between isolates B and C indicate they are from a common source, whereas isolate A originates from an independent source.

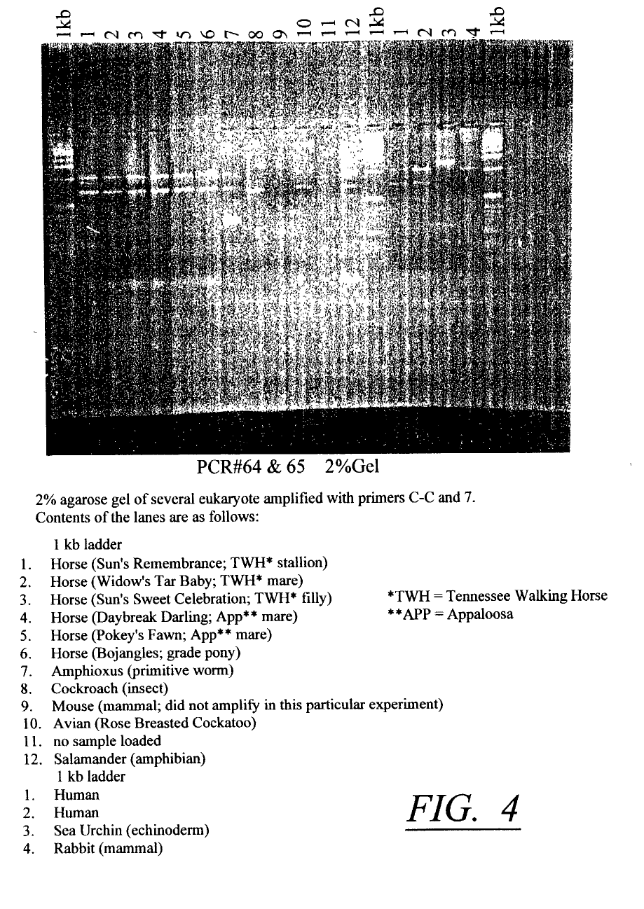

FIG. 4 is a preliminary screen of amplified intergene regions of higher life forms. PCR products were electrophoresed on a 2% agarose gel and stained with ethidium bromide.

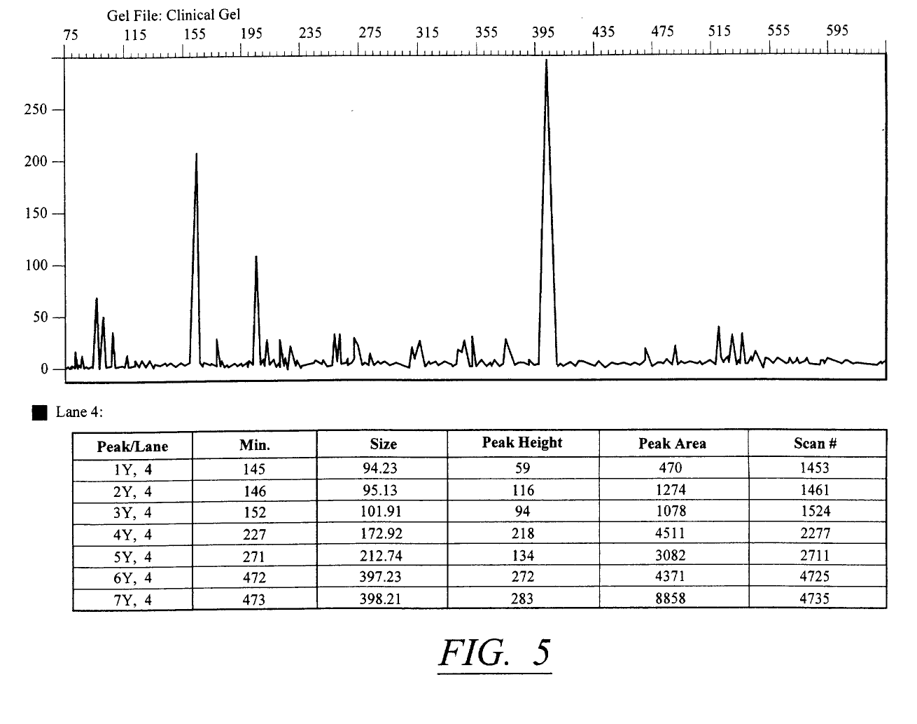

FIG. 5 is an electrophoretogram of the amplified intergene region of a horse (Daybreak Darling; TWH mare) depicted in lane four (4) in FIG. 4;

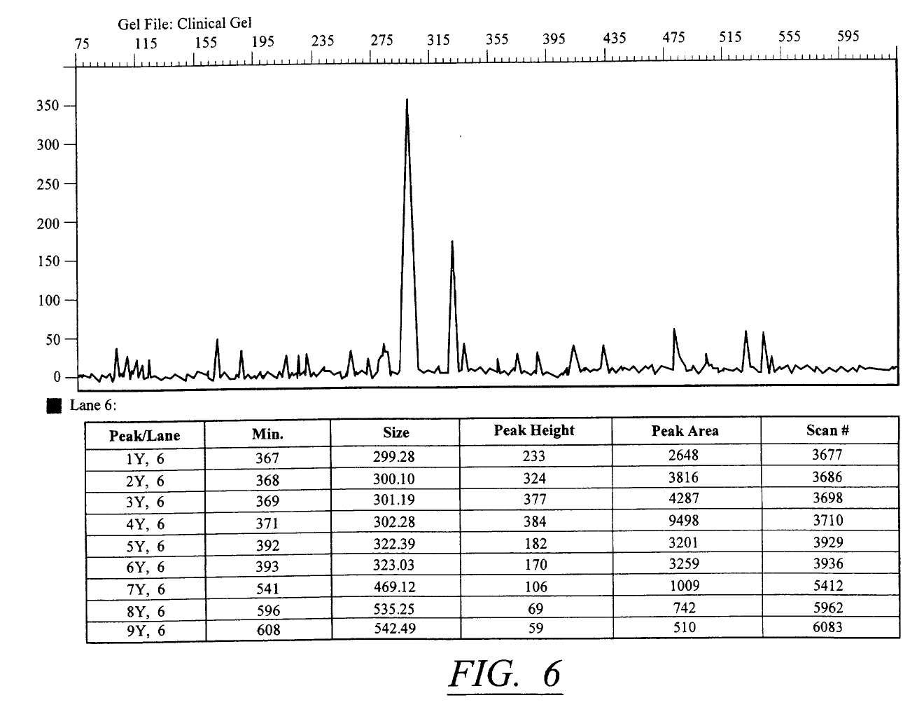

FIG. 6 is an electrophoretogram of th amplified intergene region of a horse (Bojangles; grade pony) depicted in lane 6 of FIG. 4;

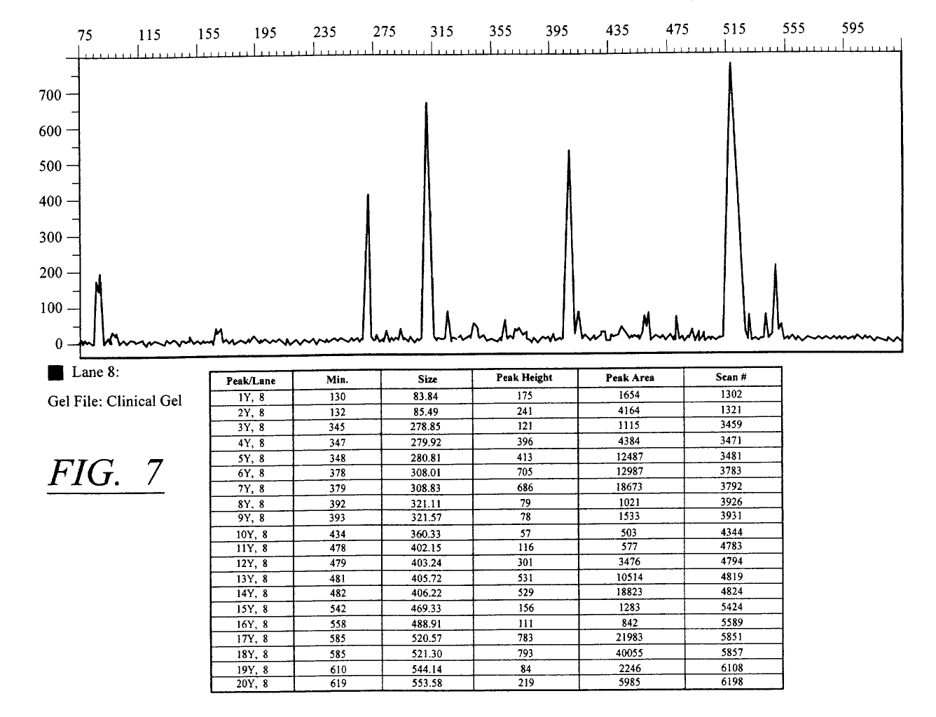

FIG. 7 is an electrophoretogram of the amplified intergene region of a cockroach (TaqI insect) depicted in lane 8 of FIG. 4;

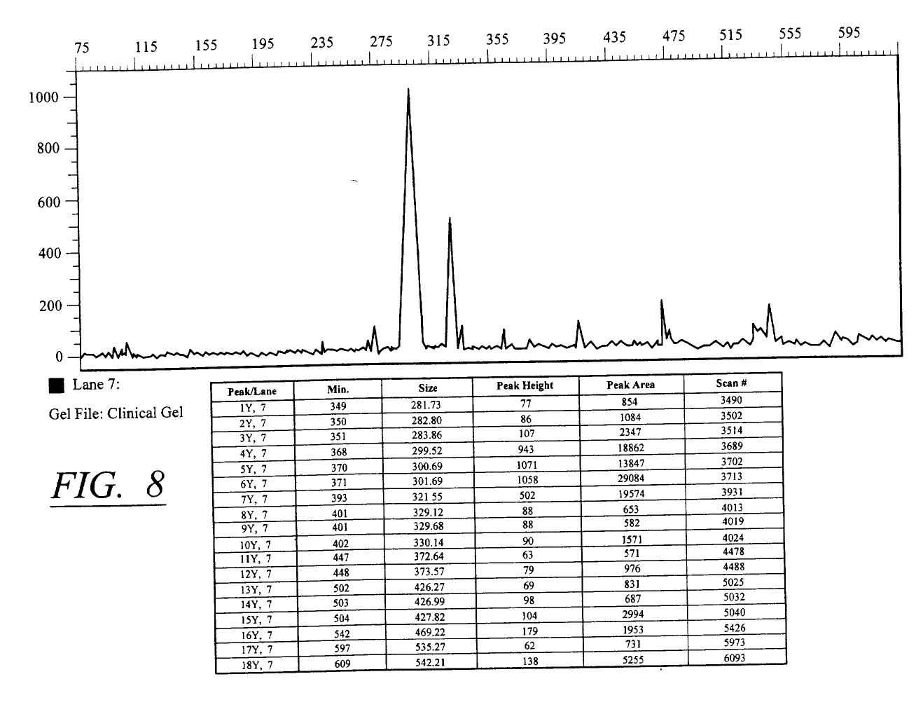

FIG. 8 is an electrophoretogram of the amplified intergene region of an Amphioxus (primitive worm) depicted in lane 7 of FIG. 4;

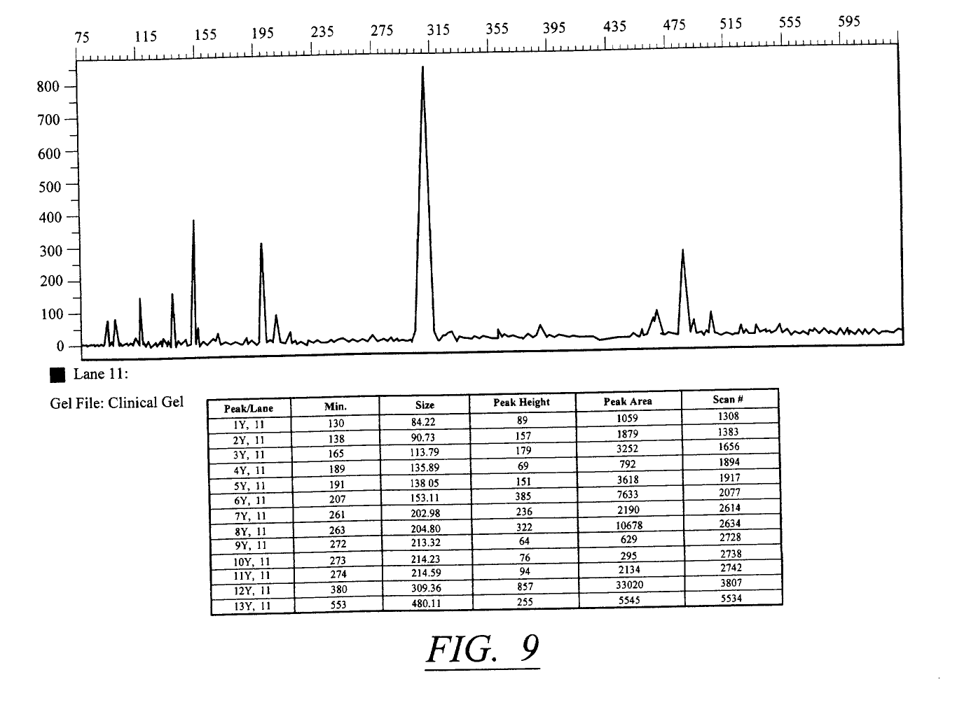

FIG. 9 is an electrophoretogram of a salamander (TaqI; amphibian) depicted in lane 12;

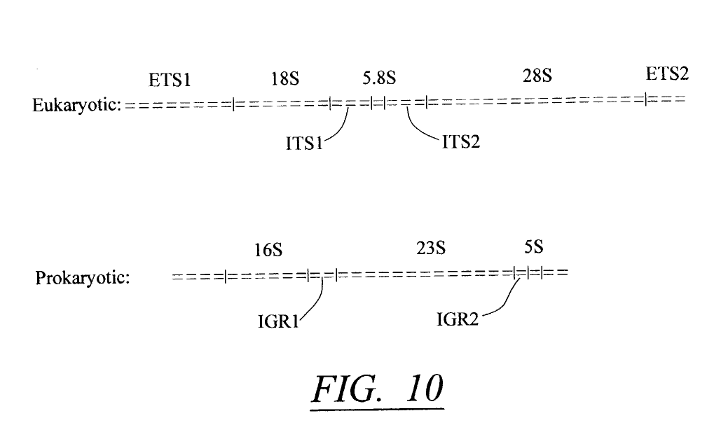

FIG. 10 is an organization of typical eukaryotic and prokaryotic rRNA gene clusters;

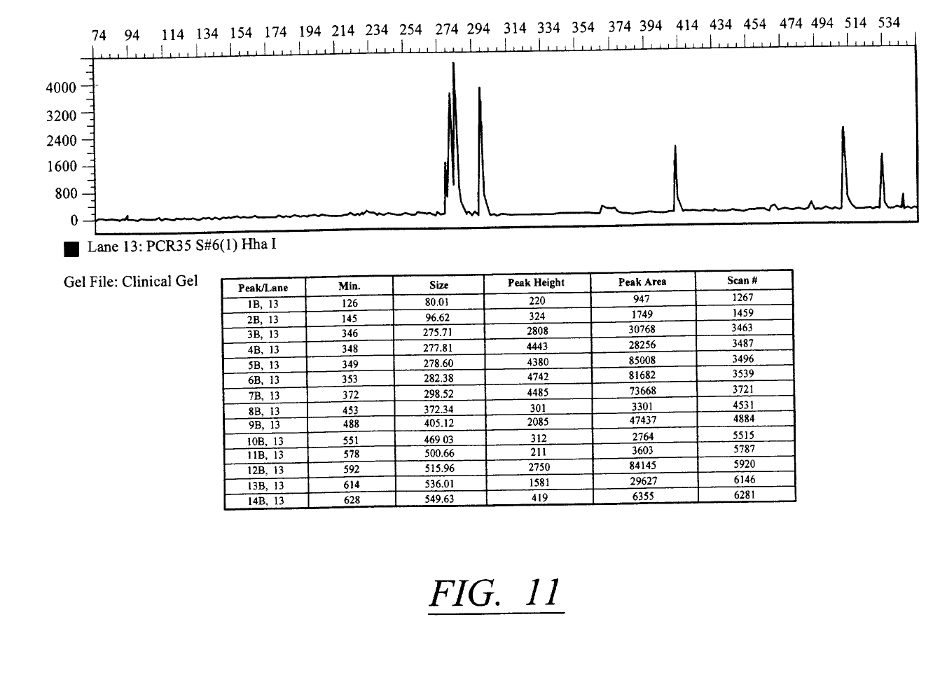

FIG. 11 is an electrophoretogram of MRSA sample #6(1) (HhaI);

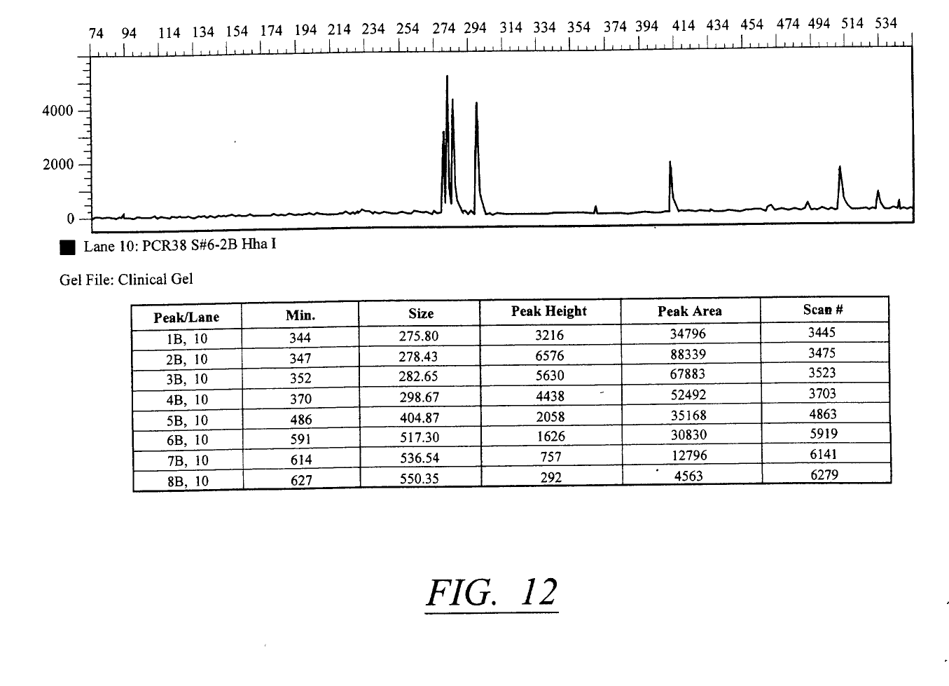

FIG. 12 is an electrophoretogram of MRSA sample #6-2B (HhaI);

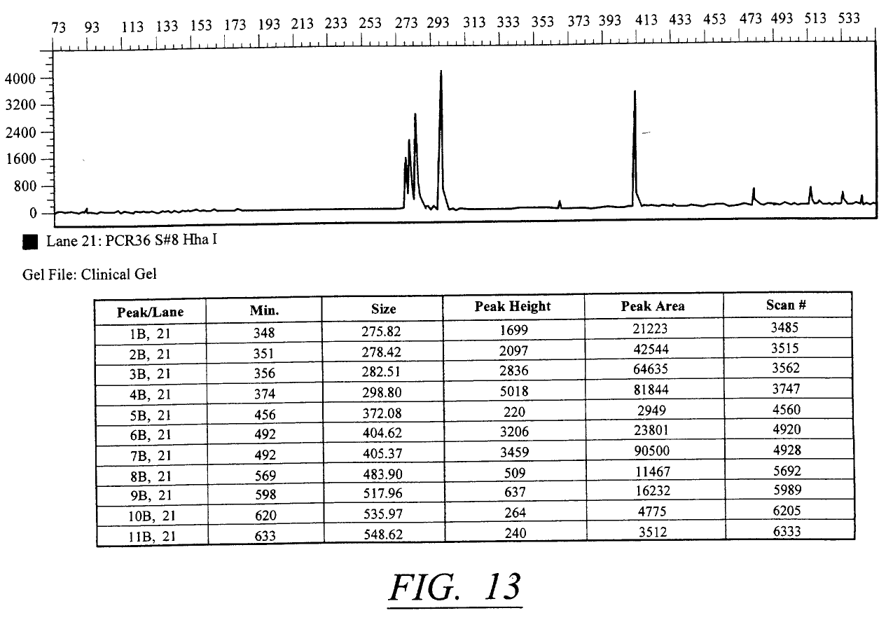

FIG. 13 is an electrophoretogram of MRSA sample #8 (HhaI);

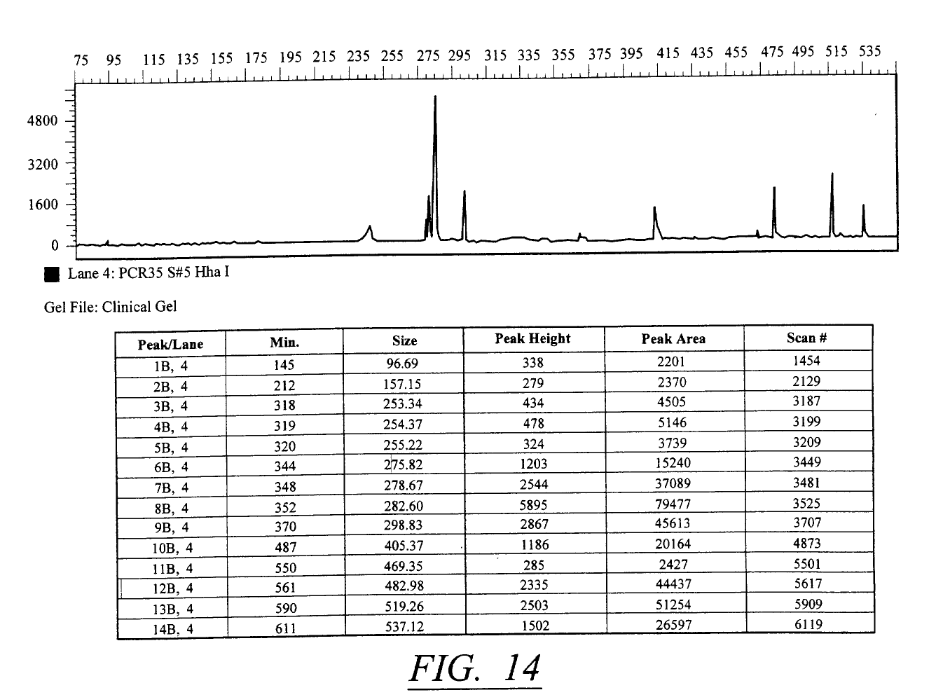

FIG. 14 is an electrophoretogram of MRSA sample #5 (HhaI);

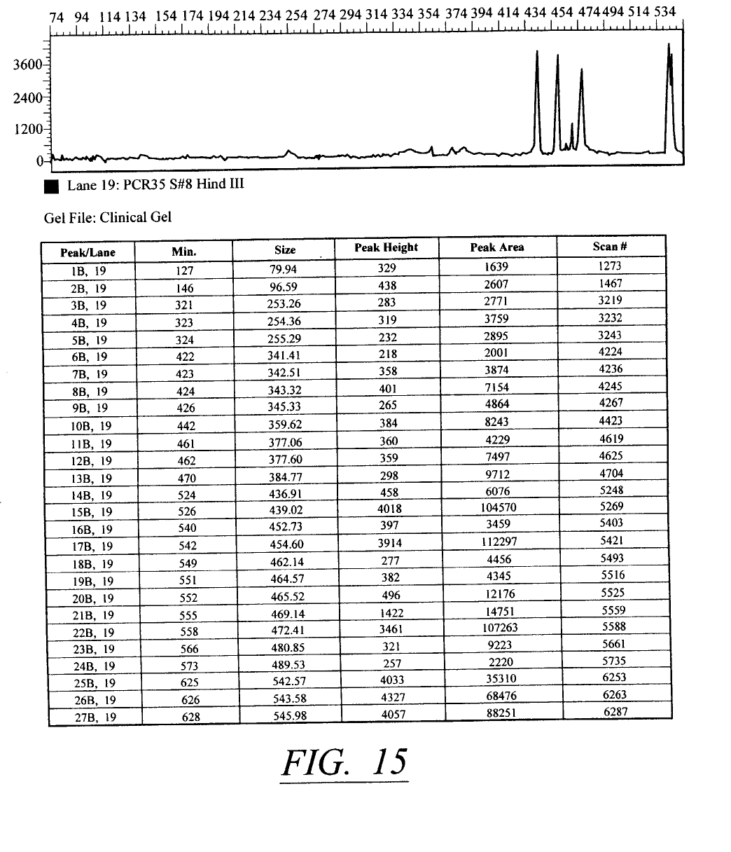

FIG. 15 is an electrophoretogram of MRSA sample #8 (Hind III); and

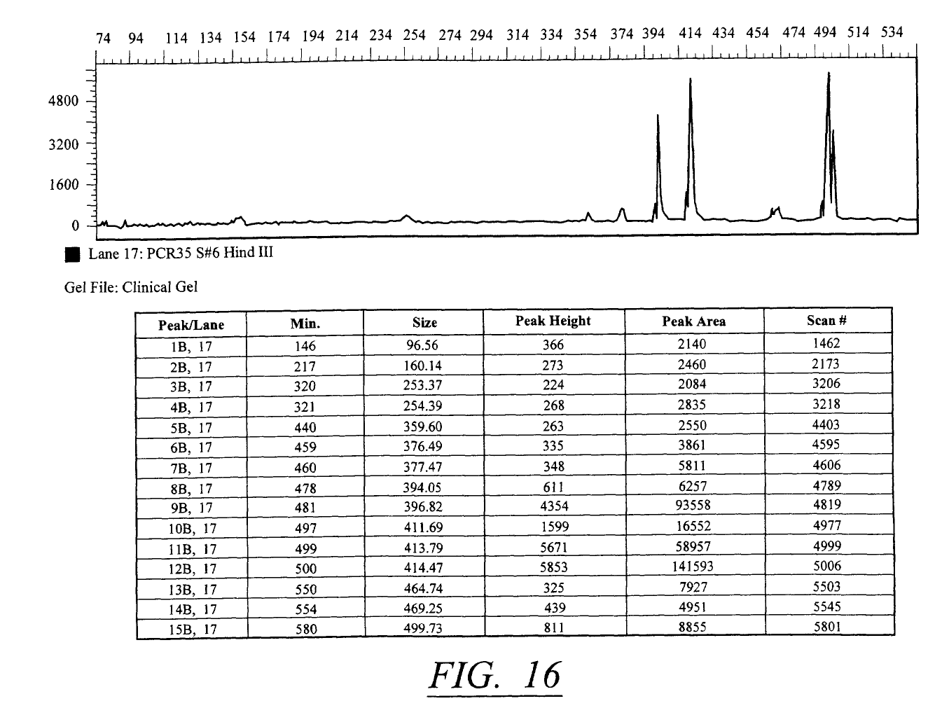

FIG. 16 is an electrophoretogram of MRSA sample #6 (Hind III);

### DETAILED DESCRIPTION

By way of illustrating and providing a more complete appreciation of the present invention and many of the attendant advantages thereof, the following detailed description is provided concerning the novel methods and kits.

The present invention provides methods and kits for DNA fingerprinting to determine similarities and/or differences between strains or individuals of subspecies. More particularly, the methods involve a rapid, semi-automated method for identifying strains of prokaryotic organisms or individuals of higher life forms and differences therebetween. More specifically, a specific DNA fragment in the rRNA intergene region is amplified by PCR using two universal oligonucleotides. While it has been realized that this area of the genome, i.e., the rRNA intergene region, is ideal for such a procedure because a highly variable spacer region of DNA is flanked by two highly conserved genes which may be used as primer sites, any area of the genome of any prokaryotic or eukaryotic life form may be selected, so long as it too contains a highly variable spacer region of DNA flanked by two highly conserved genes that may be used as primer sites for PCR amplification.

It should also be understood that any suitable oligonucleotide may serve as a primer. While a list of exemplary primers are recited in Table I, it should be understood that their complementary sequences may likewise be employed. It should also be understood that primers designated as CC and 7 in Table I are preferred and that they may be employed with those prokaryotes identified in Table II.

Exemplary of primers that may be used in accordance with this invention include those recited in Table I and the complementary sequences thereto (not listed).

TABLE I
PRIMERS
16S rRNA GENE

| | |
|---|---|
| F₂C | AGA GTT TGA TCA/C TGG CTC |
| A-C | CAG CCG CGG TAA TAC |
| G-C | AAC AGG ATT AGA TAC CCT GG |
| B-C | CAA AGG AAT TGA CGG |
| H-C | TGG CTG TCG TCA GCT CGT GT |
| E-C | GAC GTC AAG TCA TCA |
| C-C | GTA CAC ACC GCC CGT |
| T-C | AAG TCG TAA CAA GGT |
| D-C | ATT AGC TAG TAG GTG |

23S rRNA GENE

| | |
|---|---|
| 7 | TCG CTC GCC GCT ACT |
| 8 | AGG GCA TCC ACC GTG |
| 9 | ACT GGT TCA CTA TCG |
| 10 | TCG GGG AGA ACC AGC TA |
| 11 | CCA GTG AGC TAT TAC GC |
| 12 | AGG AAT ATT AAC CTG TT |
| 13 | CCA CCC TGT TGT GTC GGT TT |
| 14 | ATT TCG CTA CCT TAG |
| 15 | TTT TAT CCG TTG AGC GA |
| 16 | CTT AGA TGC TTT CAG C |
| 17 | TGA CCC ATT ATA CAA AAG GT |

5S Oligonucleotide Sequences

| | | |
|---|---|---|
| #15 | 5S | CCC ATN GGC AAC TCA |
| #323 | 5S-C | TGA GTT CGG NAT GGG |
| #810 | 5Serv | CTG TGT TCG (G/A) CA TGG |

TABLE II

Confirmed Prokaryotic Genera Which Contain
Both C—C and 7 Primer Sequences
Gram Negative Gamma Proteobacteria:

Shigella sp.
Escherichia sp.
Salmonella sp.
Klebsiella sp.
Enterobacter sp.
Citrobacter sp.
Proteus sp.
Hafnia sp.
Providencia sp.
Serratia sp.
Yersinia sp.
Vibrio sp.
Xanthomonas sp.

Gram Negative Beta Proteobacteria:

Commomonas sp.

Low GC Content Gram Positive Genera:

Bacillus sp.
Staphylococcus sp.
Streptococcus sp.

High GC Content Gram Positive Genera:

Arthrobacter sp.
Terrabacter sp.
Cyanobacterium Genera

In carrying out the methods of the present invention, the amplified product is preferably labeled during or prior to amplification. This may be accomplished by using one 5′ end labeled primer from the 16S gene for prokaryotic typing. Several other options, however, may be employed. For example, 1.) a 5′ end labeled primer may be employed from the 23S gene or 5S gene, 2.) 5′-end-labeled primers may be employed from the 16S and 23S or 23S and 5S or 16S and 5S genes, 3.) both primers may be employed in conjunction with fluorescently labeled dioxynucleotide triphosphate (dNTPs), or 4.) labeled dNTPS may be employed alone in the PCR reaction. With respect to eurokaryptic typing, similar combinations may be used, but for the eukaryotic structural genes, i.e., 18S or 5.8S or 28S, or 18S and 5.8S, or 5.8S and 28S, or 18S and 28S. In either prokaryotic or eukaryotic typing, it should be understood that labeling may be accomplished by using both 5′ and labeled primers in conjunction with fluorescently labeled deoxynucleotide triphosphates (dnTPS) or by using the labeled dNTPs alone in the PCR reaction.

It should be further appreciated that while amplification of the highly variable spacer region between the 16S and 23S genes is preferred, the methods of the present invention contemplate amplifying highly variable spacer regions between the 23S and 5S genes in prokaryotes and the highly variable spacer regions between the 18S and 5.8S and the 5.8S and 28S genes in eukaryotes. Of course, the present invention contemplates amplifying any highly variable spacer region of DNA or RNA which lies between two highly conserved DNA or RNA regions, respectively.

To type prokaryotes, such as bacterial strains, clinical isolates are sent from a hospital (or other source) on agar slants. Multiple colonies (4-5) are collected on a loop and resuspended in 50 μl 10 mM EDTA in a microcentrifuge tube. The tube is boiled for five minutes and the resultant lysate, containing the DNA template, is used in a polymerase chain reaction (PCR).

PCR samples are prepared using 10 μl of a 1033 Taq Amplification Buffer (50 μl 1 M Tris-HCl, pH 8.3; 250 μl 1 M KCl; 20 μl 1 M MgCl₂; 2.25 μl NP-40; 2.25 μl Tween 20; 5 μl 1% gelatin; and 170.5 μl distilled water), 4 μl 2.5 mM dNTP mix (2.5 mM each dATP, dCTP, dGTP, dTTP), 100 ng fluorescently labeled 16S rRNA primer (GTA CAC ACC GCC CGT), 100 ng nonlabeled 23S rRNA primer (TCG CTC GCC GCT ACT), 1-10 μl of lysate from whole cells, 2.5 units AmpliTaq DNA polymerase, and sterile distilled water to bring the total volume

to 100 μl. This mixture is overlayed with 100 μl mineral oil and placed in a Thermal Cycler (Perkin-Elmer, Norwalk, Conn.) under the following reaction conditions: 5 minute delay at 95° C. for 40 seconds, 50° C. for 25 seconds, 72° C. for 3 minutes; 10 minute delay at 72° a 4° C. soak.

The PCR mixture is then separated from the oil overlay and 8 μl of the aqueous phase is mixed with 2 μl of a standard gel loading buffer. The aliquot is electrophoresed on a 2% agarose gel at 70 volts for one to two hours. The gel is stained with ethidium bromide and viewed under U-V illumination. The banding pattern reflects the rRNA gene cluster number of each bacterium. This initial pattern can be used as a preliminary test to distinguish between bacterial strains. If the banding patterns are very similar and more definitive results are desired, then a fingerprint is one on an automated sequencer.

The PCR mixture is passed through a G-50 column to remove unincorporated dNTPs and primers. The DNA concentration is determined on a minifluorometer. The PCR mixture then undergoes a 2-hour restriction digest with various four to six base endonucleases. An aliquot of the restriction digests is loaded onto a 6% polyacrylamide gel and electrophoresed on the 373A DNA Sequencing System (Applied Biosystems, Inc., Foster City, Calif.). The resulting DNA fingerprint is preserved in a gel file and can be printed onto paper as waveform patterns. See waveform FIGS. GeneScan software, which has been introduced by ABI, should enable the determination of DNA fragment size and gene dosage.

The Genescan software automates DNA fragment analysis by using an internal lane size standard on either an agarose or polyacrylamide gel to determine DNA fragment size and quantity. The software is used with either the 373A Electrophoresis unit (Applied Biosystems, Inc., Foster City, Calif.) or with the ABI horizontal gel unit (Gene Scanner 362 Flourescent Fragment Analyzer). Both electrophoresis units use an argon laser to excite fluorescent dyes which have been attached to the DNA molecules. The fluorescence emission is detected by a photomultiplier tube which converts the light signal to a digitized signal. The data are collected in real time on a Macintosh computer.

This technology allows to quantitatively analyze DNA fragments in just a few hours, as opposed to the lengthy turnaround time associated with the traditional methods of Southern hybridizations and Dot Blots. Moreover, these traditional protocols generally require a week to complete and are labor-intensive. In addition, radiolabeled nucleotides must be used during analysis.

In general, procedures for certain aspects of the methods of the present invention are 5 similar to that followed for automated sequencing. A 6% polyacrylamide gel is prepared and the gel is cast with a 24- or 36-well comb specifically designed for use with the Genescan software. DNA which has been 5′ end labeled and digested with a restriction endonuclease is dried in a SpeedVac concentrator (Savant), resuspended in a mix of 4 μl internal lane size standard (GENESCAN-2500 ROX or FAM), and is located onto the gel. The 24 cm well to read gel is electrophoresed, for example, for 14 hous in TBE buffer under a constant current of 2500 volts.

Fragment analysis is carred out with the matrix configuration determined for standard DyeDeoxy Terminator sequencing reactions or with a custom marix. The matrix is used in the algorithms which calculate DNA mobility through the gel. Essentially, a waveform pattern (electrophoretogram) and tabular data are generated for each sample loaded. The electrophoretogram is a record of the banding pattern within the gel. Signal intensity follows a baseline value until a band is distinguished. A peak is present on the electrophoretogram for that DNA sample.

Tabular data are also generated for each sample. These data are presented in a spreadsheet format and given numerical value to each DNA band detected. Each band (peak) is numbered and the time at which that peak was detected is recorded. The size of each bank is calculated according to the internal lane size standard. Although several methods are available for determination of fragment size, the Local Southern method is sued because fragments generally in the 50 to 400 basepair range are analyzed. It is believed that this method creates a best-fit line through all the available points and then uses value on that line to calculate fragment values. Peak height and peak area are also included in the tabular data. These values, particularly peak area, are important in quantitating DNA samples and in determining gene dosage.

Because several fluorescent dyes are detected on either of the electrophoresis units currently sold by ABI, four different labels may be used to analyze DNA fragments within one lane on the gel. The number of samples analyzed on each gel may be increased, which therefore increases throughout by a factor of three or four. In one instance, it may be preferred to analyze a sample using three different restriction endonucleases. Therefore, a labeled 16S primer, for example, would be used in the three different digests but with a different colored label associated with each particular digest; perhaps blue with HhaI, yellow with HindIII, and green with TaqI. Following the restriction digest, the samples would be combined and loaded onto the gel. A multicolored banding patter would result and would ultimately provide a very specific fingerprint for that particular isolate. This ability also allows for a large number of samples in a short period of time. For example, up to 96 resriction patterns could be determined on one gel run with 24 lane run or 144 patterns on a 36 lane gel.

ABI is believed to be currently creating an upgrade which would permit gel runs as short as one to two hours. With this upgrade, two or three gels could be run within an eight hour work day and an additional gel could be run overnight. A laboratory remaining open for 24 hours could conceivably process ten to twelve gels a day per nstrument. Therefore, 700 to 1100 fingerprints could be generated every 24 hours per insrument.

In eukaryotic cells, the ribosomal RNA (rRNA) genes are organized somewhat differently than in prokaryotic (bacterial) cells. Most notably the 5S genes, which are closely linked to (and transcribe with) the other rRNA genes in bacteria, are separated from the 18S and the 28S rRNA genes in eukaryotic cells. In addition the ribosomes of eukaryotic cells contain an extra small RNA, the 5.8S RNA which has been shown to be homologous to the 5′-end of the bacterial 23S RNA. The 5.8S gene is located just in front of (on the 5′ side of) the 28S gene. Shown below in FIG. 10 is the organization of a tpical eukaryotic rRNA gene cluster. It is compared to the homologous cluster of rRNA genes observed in almost all bacteria.

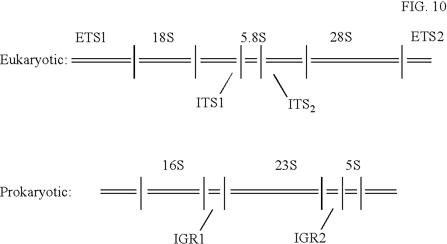

The prokaryotic rRNA gene cluster (rrn) is composed of the 16S RNA gene followed by the 23S and 5S genes. The two IGRs are highly variable interGene regions between the three structural genes. They are transcribed with the genes to give a precursor RNA of about 5200 nucleotides, but are removed by ribonuclease cutting of the total transcript. A promoter of transcription is located just before the 16S gene and a transcriptional terminator is located Just beyond the 5S gene.

In eukaryotes a much larger initial transcript is made—typically about 13,000 nucleotides long. Before the 18S gene is ETS1, the first external transcribed spacer. A second external space is located just beyond the 28S gene. Two highly variable internal transcribed spacers, ITS1 and ITS2, are located between the 18S and 5.8S and between the 5.8S and 28S genes, respectively. There are approximately 2300 nucleotides in the 18S RNA, 160 nucleotides in the 5.8S RNA and some 4200 nucleotides in the 28S RNA. In all eukaryotes multiple copies of this rRNA gene cluster are found in the nucleolus, and there may be several nucleoli inside the nucleus of a single cell. In the nucleolus the rRNAs are transcribed, matured (cut) and assembled (along with the 5S RNA and 80 proteins) into ribosomes.

The number or gene dosage of these rRNA clusters in eukaryotic cells varies from as few as 100 to over 1500 copies per genome. Yeast has 140 copies, Neurospora has 200, Drosophilia has 240 and the human genome contains about 450. They are found as long repetitive units (the nucleolar organizer region) where as many as 500 copies of the entire cluster are repeated in tandem. In humans nucleolar organizers are found on the end of the short arms of chromosomes 13, 14, 15, 21, and 22.

Typical components of kits of the present invention include:

1. PCR Master Mix

a. 10× Amplification Buffer

b. 2.5 mM dNTP mix (labeled or unlabeled)

c. two primers, wherein at least one primer is preferably labeled:

2. DNA Polymerase;

3. Restriction endonucleases; and

4. Pre-packed G-50 columns.

In practicing the methods and utilizing the kits of the present invention, the following laboratory equipment may be required: automated sequencer, Thermal Cycler, Agarose Gel Electrophoresis Apparatus, Clinical Centrifuge and heating blocks or water baths.

The present invention will now be further illustrated with reference to the following Examples.

### EXAMPLES

### Example 1

MRSA (methicillin resistant Staphylococcus aureus) isolates are identified at the hospital and delivered on typticase-soy agar slants. The patient name and hospital identification number are recorded and an isolate number is designated. Each number assigned has a three-letter prefix designating the hospital or origin (AMH, Archbold Memorial Hospital; TMH, Tallahassee Memorial Hospital) as well as a sequential numerical designation.

Five colonies from the slant are collected on a 5 millimeter bacterial inoculation loop and resuspended in 50 μl 10 mM EDTA in a microcentrifuge tube. The tube is boiled for 5 minutes and the resultant lysate, containing the DNA template, is used in a polymerase chain reaction (PCR). Although it is preferable to use a 5- end labeled primer from the 16S gene for prokaryotic typing, several alternative options are also available for strain typing. These include:

(1) using a 5′ end labeled primer from the 23S gene; (2) using both the 5- end labeled 16S and 23S primers; (3) using both 5′ end labeled primers in conjunction with fluoresenctly labeled deoxynucleotide triphosphates (dNTPs); and/or (4) by using the labeled dNTPs alone in the PCR reaction.

PCR reaction mixture is prepared using 10 μl of a 10× Taq Amplification Buffer (50μ of a 10× Taq Amplification Buffer (50 μl 1 M Tris-HCl, pH 8.3; 250 μl 1 M MgCl₂; 2.25 μl NP-40; 2.25 μl 1% gelatin; and 170.5 μl distilled water), 4 μl 2.5 mM dNTP mix (2.5 mM each dATP, dCTP, dGTP, dTTP), 100 ng fluorescently labeled 16S rRNA primer (GTA CAC ACC GCC CGT), 100 ng nonlabeled 23 S rRNA primer (TCG CTC GCC GCT ACT), 1-10 μl of lysate from whole cells, 2.5 units AmpliTaq DNA polymerase, and sterile distilled water to bring the total volume to 100 μl. This mixture is overlayed with 100 μl mineral oil and placed in a Thermal Cycler (Perkin-Elmer, Norwalk, Conn.) under the following reaction conditions: 5 minute delay at 95° C. for 40 seconds, 50° C. for 25 seconds, 72° C. for 3 minutes; 10 minute delay at 72° C., followed by a 4° C. soak.

The PCR mixture is then separated from the oil overlay and 8 μl of the aqueous phase is mixed with 2 μl of a standard gel loading buffer. The aliquot is electrophoresed on a 2% agarose gel at 70 volts for one to two hours. The gel is stained with ethidium bromide and viewed under U-V illumination. The banding pattern reflects the rRNA gene cluster number of each bacterium. This initial pattern can be used as a preliminary test to distinguish between bacterial strains. If the banding patterns are very similar and more definitive results are described, then the fingerprint is done on an automated sequencer.

The PCR mixture is passed through a G-50 column to remove unincorporated dNTPs and primers. The DNA concentration is determined on a miniflurometer. The PCR mixture then undergoes a 2-hour restriction digest with various four or six base endonucleases. Hhal and HindIII are generally preferred for restriction digests. However, other restriction endonucleases may prove to be more informative for other species. 100 ng of the amplified DNA product is added to a tube contianing 1 μl React Buffer (Gibco BRL, Grand Island, N.Y.), and sterile distilled water to a volume of 9 μl. One microliter restriction endonuclease (HhaI or HindIII) is added and the mixture is incubated at 37° C. for a minimum of two hours. The entire restriction digest is dried in SpeedVac Concentrator (Savant) and each is resuspended in a mixture of 4 μl deionized formamide and 0.5 μl GS2500P internal lane size standard (ABI, Foster City, Calif.). The samples are loaded onto a 6% polyacrylamide gel and electrophoresed on the 373A DNA Sequencing System (Applied Biosystems, Inc., Foster City, Calif.) under the Genescan software. The resulting DNA fingerprint is preserved in a gel file and can be printed onto paper as waveform patterns (electrophoretograms). See FIGS. 11-16.

### Example II

Eukaryotic Fingerprinting in the Ribosomal Gene Clusters

Fingerprint patterns of high life forms have been created in accordance with Example II from the ribosomal gene clusters using the same priming sites as those used for prokaryotic (bacterial) material typing protocol. The priming sites for C-C and 7 are believed to be conserved in those organisms whose ribsomal intergene region amplified. These higher life forms included horse, amphioxus (the first evolutionary organism to develop a notocord), cockroach (insect), sea urchin (echinoderm), salamander (amphibian), Rose Breasted Cockatoo (avian), rabbit (mammal), and human (mammal). As show in FIG. 4., which is a photograph of an agarose gel, illustrate th PCR amplifications for the organisms used in our study. FIGS. 5-9 depict the waveform patterns for the horses in lanes 4 and 6, cockroach in lane 8, amphioxus in lane 7 and salamander in lane 11 of FIG. 4.

It is also believed that other higher life forms seem to have the primer 7 sequence conserved. These include Physarum polycephalum (slime mold), mucor racemosus (dimorphic fungus), Citrus limon (lemon tree), Mus musculus (mouse), Drosophila melanogaster (fruitfly), Tritrichomonas foetus (protozoan), and Homo sapien (human). It is also believed that the primer 7 site is located in the 5′ end of the 28S gene, rather than in the 5.8S gene as we previously believed. However, a small portion of the primer 7 sequence (approximately 6 bases) was found in the 3′ end of the 5.8S sequence of Tritrichomonas foetus. Therefore, the amplified fragments presumably include a large and small band; the large band consisting of a small portion of the 18S gene (the primary site o C-C), internal transcribed space 1 (ITS1), the 5.8S RNA gene, internal transcribed space 2 (ITS2), and a small portion of the 28S RNA gene (the primary site of 7) while the smaller band contains the small portion of the 18S gene, ITS1, and most of the 5.8S gene.

The amplified DNAs from the above higher life forms were digested with HhaI and HindIII (the endonucleases used in the prokaryotic study), but found that these sites were relatively uninformative. Other restriction endonucleases which may prove to be better candidates for generating fingerprint patterns have been identified. For example, it is believed that the restriction endonucleases TaqI, SspI, NedI, NdeII, and RsaI may enable one skilled in the art to identify individuals within a subspecies for possible linkage and pedigree studies. It is therefore believed that the selection of proper restriction endonucleases will enable those skilled in the art ot identify individuals within a subspecies of higher life forms.

Data of the fingerprint patterns generated relative to the higher life forms is recited in FIGS. 4-9. This data was generated using flourescent dUTP, rather than a labeled primer. In comparing the data between “Daybreak darling TaqI” and “Bojangles TaqI,” which concern two horses, a Tennessee Walking Horse and a grade pony, these two individuals seem to have unique fingerprint patterns, but further confirmation is believed to be needed.

The following constitutes a summary of the procedures utilized to generate the data relative to the higher life forms. A fresh deoxynucleotide triphosphate solution was prepared from stock and contained one-half the normal dTTP concentration. The final nucleotide concentrations within the dNTP solution were 25 mM each dGTP, dATP, and dCTP and 12.5 mM dTTP.

PCR was performed on blood samples by first treating the whole blood with GeneReleaser (BioVentures, Inc., Murfreesboro, Tenn.) as per manufacturer's protocol. PCR reaction mixture was then overlayed and PCR commenced. All other genomic DNA samples were kindly provided and previously prepared by Dr. deKloet and Gus Ray at Florida State University.

A polymerase chain reaction was prepared with approximately 50 ng template DNA, 10 μl 10× amplification buffer (50 μl 1 M Tris-HCl, pH 8.3; 250 μl M KCl; 20 μl M MgCl₂; 2.25 μl NP-40; 2.25 μl Tween 20; 5 μl 1% gelatin; and 170.5 μl distilled water), 4 μl of the prepared dNTP solution (final concentration 2.5 mM each dGTP, and dCTP and 1.25 mM dTTP), 1 μl of a 1:10 FluoroRed Dilution (Rhodamine-4-dUTP, Amershal Life Sciences, Arlington Heights, Ill.), 0.5 μl each amplification primers, 2.5 units Amplitaq DNA polymerase (Perkin-Elmer, Norwalk, Conn.), and sterile distilled water to bring the total volume to 100 μl. PCR was carried out in a Thermal Cycler (Perkin-Elmer) under the following reaction conditions: 5 minute delay at 95° C.; 95° C. denaturation for 40 seconds, 50° C. annealing for 25 seconds, and 72° C. extension for 3 minutes for 30 cycles, followed by a 10 minute delay at 72° C.

The PCR mixture was then separated from the oil overlay and 8 μl of the aqueous phase was mixed with 2 μl of a standard gel loading buffer. The aliquot was electrophoresed on a 2% agarose gel at 70 volts for one to two hours. The gel was stained with ethidium bromide and viewed under U-V illumination. The CR mixture was passed through a G-50 column to remove unincorporated dNTPs and primers and the DNA concentration was determined on a minifluorometer. The PCR mixture than underwent a 2-hour restriction digest with various four or six base endonucleases. It is believed that TaqI and SspI may prove to be the most informative for the higher life forms in this study; however, other restriction endonucleases may prove to be more informative for other species. 100 ng of the amplified DNA product was added to a tube containing 1 μl React Buffer (Gibco BRL, Grand Island, N.Y.) and sterile distilled water to a volume of 9 μl. One microliter restriction endonuclease (TaqI or SspI) was added and the mixture was incubated at 37° C. for a minimum of two hours. The entire restriction digest was dried in a SpeedVac Concentrator (Savant) and each resuspended in a mixture of 4 μl deionized formamide and 0.5 μl GS2500P internal lane size standard (ABI, Foster City, Calif.). The samples were loaded onto a 6% polyacrylamide gel and electrophoresed on the 373A DNA Sequencing System (Applied Biosystems, Inc., Foster City, Calif.) under the Genescan software.

GeneReleaser (BioVentures, Inc., Murfreesboro, Tenn.), is a commercially available reagent which releases DNA form whole blood, tissues, and bacterial cultures while chelating polymerase inhibitors released during cell lysis. GeneReleaser obviates genomic DNA purification and is claimed to improve amplification yied and specificity. It is provided as a ready to use stock and releases genomic DNA in ten minutes.

While the manufacturer's instructions call for 1 μl of whole bood, it is believed that by using 3 μl of whole bood, the yields are increased considerably. Therefore, 3 μl whole bood is mixed with 20 μl GeneReleaser in a 0.5 ml microfuge tube. The mix is overlayed with 100 μl mineral oil and the reaction is performed under the following condition in a Thermocycler (Perkin Elmer, Norwalk, Conn.):

| | |
|---|---|
| 65° C. | 30 seconds |
| 8° C. | 30 seconds |
| 65° C. | 90 seconds |
| 97° C. | 180 seconds |
| 8° C. | 60 seconds |
| 65° C. | 180 seconds |
| 97° C. | 60 seconds |
| 65° C. | 60 seconds |
| 80° C. | hold. |

The amplification reagents are added under the oil without mixing the tube contents and amplification is executed as per our prakaryote protocol.

The present invention may, of course, be carried out in other specific ways than those herein set forth without departing from the spirit and essential characteristics of the invention.

The present embodiments are, therefore, to be considered in all respects as illustrative and not restrictive and all changes coming within the meaning and equivalency range of the appended claims are intended to be embraced herein.

Having described our invention.

What is claimed is:

1. A method for typing a cell, the method comprising the steps of:
(a) providing a cell of unknown type, the cell of unknown type having a nucleic acid forming a portion of the cell's genome selected from the group consisting of IGR1, IGR2, ETS1, and ETS2;
(b) isolating the nucleic acid from the cell of unknown type;
(c) amplifying the nucleic acid by PCR to form a PCR product;
(d) digesting the PCR product with a restriction endonuclease to form a plurality of restriction fragments;
(e) separating the plurality of restriction fragments to generate a restriction pattern; and
(f) comparing the restriction pattern generated in step (e) with a restriction pattern of a cell of known type.

2. The method of claim 1, wherein the step (a) of providing the cell of unknown type comprises isolating the cell of unknown type from a patient.

3. The method of claim 1, wherein the step (c) of amplifying the nucleic acid by PCR to form a PCR product is performed under conditions that result in the PCR product being fluorescently labeled.

4. The method of claim 1, wherein the restriction enzyme is selected from the group consisting of four-base endonucleases and six-base endonucleases.

5. The method of claim 1, wherein the restriction enzyme is selected from the group consisting of Hhal and HindIII.

6. The method of claim 1, wherein the cell is a prokaryotic cell.

7. The method of claim 6, wherein the prokaryotic cell is a bacterium.

8. The method of claim 1, wherein the cell is a eukaryotic cell.

9. The method of claim 1, wherein the PCR product comprises a polynucleotide having a 5′ nucleotide and a 3′ nucleotide and wherein the plurality of restriction fragments comprise both a first polynucleotide fragment containing the 5′ nucleotide and a second polynucleotide fragment containing the 3′ nucleotide.

10. The method of claim 1, wherein the step (e) of separating the plurality of restriction fragments to generate a restriction pattern is performed by electrophoresis.
